(19) United States
(12) Patent Application Publication
(10) Pub. No.: US 2024/0122662 A1
(43) Pub. Date: Apr. 18, 2024

(54) METHOD FOR CONTROLLING A LIMITED TELEOPERATION, OVER A SUBSET OF DEGREES OF FREEDOM, OF A MASTER-SLAVE ROBOTIC SYSTEM FOR MEDICAL OR SURGICAL TELEOPERATION AND RELATED ROBOTIC SYSTEM

(71) Applicant: MEDICAL MICROINSTRUMENTS S.P.A., Calci, Pisa (IT)

(72) Inventors: Matteo Tanzini, Pisa (IT); Emanuele Ruffaldi, Pisa (IT); Massimiliano Simi, Pisa (IT); Giuseppe Maria Prisco, Pisa (IT)

(21) Appl. No.: 18/546,219

(22) PCT Filed: Feb. 14, 2022
(86) PCT No.: PCT/IB2022/051279

§ 371 Date: Aug. 11, 2023

(30) Foreign Application Priority Data
Feb. 16, 2021 (IT) 102021000003431

(51) Int. Cl. A61B 34/35; A61B 34/00

(52) U.S. Cl. CPC A61B 34/35; A61B 34/74; A61B 2090/0818

(57) ABSTRACT

A method controls a robotic system for medical or surgical teleoperation. The robotic system includes a hand-held master device, mechanically unconstrained to the ground and moveable by an operator; and a slave device including a surgical instrument adapted to be controlled by the master device, so that movements of the slave device, or of the surgical instrument of the slave device, referred to one or more of a plurality of controllable degrees of freedom are controlled by respective movements of the master device, according to a master-slave control architecture. The method firstly includes the steps of defining a first enslaved state of the system and a second decoupled state of the system. A controller controls transitions between the first and second states. The controllable degrees of freedom include degrees of freedom of translation and of orientation. A master-slave robotic system for medical or surgical teleoperation performs the method.

### TECHNOLOGICAL BACKGROUND OF THE INVENTION

### Field of Application

The present invention relates to a method for controlling a limited teleoperation, over a subset of degrees of freedom, of a master-slave robotic system for medical or surgical teleoperation, and a corresponding master-slave robotic system for medical or surgical teleoperation equipped so as to perform the aforesaid method.

### Description of the Prior Art

In the field of master-slave robotic systems for medical or surgical teleoperation, the use of scale reduction factors between the movement in position of the master device and the slave device is known, as well as the entry and exit into/from specific machine states in which some of the degrees of freedom are temporarily locked or decoupled to allow repositioning the master devices themselves in the center of the workspace thereof for a more comfortable position or to reach the edges of the slave workspace.

In particular, in such a context, master consoles are known with a mechanically constrained and motorized attachment which acts as a master controller device. In such cases, to allow an easy return from a state of partial teleoperation to one of complete teleoperation, in the partial teleoperation step the master-slave orientation is constantly kept aligned by locking the degrees of orientation of the master and in some cases also by moving the master device by motors so as to ensure the full correspondence of the orientation of the master with that of the slave device for the entire duration of the partial teleoperation.

Solutions have recently emerged with master devices not mechanically constrained to the console of the robotic system, i.e., unconstrained or “ungrounded” or “flying”, i.e., of the type as shown for example in documents WO-2019-020407, WO-2019-020408, WO-2019-020409 to the same Applicant, as well as of the type as shown for example in document U.S. Pat. No. 8,521,331.

Therefore, the problem remains of how to ensure, even in the case of unconstrained or “flying” master devices, an initial step of preparation for teleoperation, in which a satisfactory level of initial alignment must be reached between master and slave devices, while allowing the operator (for example, physician or surgeon) to place himself in an initial position suitable for teleoperation. In such a step, the movements of the master device must obviously be decoupled from the movements of the slave device.

Such a feature is highly important, since it is linked to highly stringent safety requirements which must be respected by the robotic system.

Furthermore, the surgeon has comfort and practicality needs, especially when the scaling between the movements of the master device and the slave device is high (i.e., when, as occurs in micro-surgery applications, the “slave” movement is smaller than the “master” movement, scaled by a factor between 5 and 20, for example).

In such a case, it can occur that while holding the master devices, the surgeon must move his hands very widely, and may even reach the edge of the workspace of the master device with his hands (similarly, consider the movement of a mouse on a pad, in conditions where the mouse reaches the edge of the pad without having yet brought the cursor to the edge of the screen).

Therefore, there are numerous reasons requiring a decoupling between master device and slave device, and a dedicated method which manages the initial step of preparation for teleoperation.

However, such a temporary decoupling step entails the fact that the slave device does not follow the master device, and therefore, as long as the master and slave are decoupled, it is not possible to ensure a satisfactory alignment of master device and slave device, which is a serious drawback in terms of maneuverability of the teleoperation system and safety for the patient.

Therefore, in the field of master-slave robotic systems for medical or surgical teleoperation, there is a strong need to carry out auxiliary teleoperation procedures, which on the one hand allow ensuring the absolute safety of the patient and the surgeon's comfort while operating, and on the other hand allow effectively obtaining the master-slave alignment, at the end of the initial preparation step and before the beginning of the actual teleoperation as well as in the context of transitions from and/or towards a limited teleoperation state.

### SUMMARY OF THE INVENTION

It is the object of the present invention to provide a method for controlling a robotic system for medical or surgical teleoperation, which allows at least partially overcoming the drawbacks claimed above with reference to the prior art, and responding to the aforementioned needs particularly felt in the technical field considered. Such an object is achieved by a method according to claim 1.

Further embodiments of such a method are defined in claims 2-16.

It is also an object of the present invention to provide a robotic system for medical or surgical teleoperation equipped to perform the aforesaid control method. Such an object is achieved by a method according to claim 17.

Further embodiments of such a system are defined by claims 18-37.

By virtue of the proposed solutions, it is possible to ensure a certain satisfactory level of master-slave alignment during a limited teleoperation state as well as during state transitions from and/or towards said limited teleoperation state.

### BRIEF DESCRIPTION OF THE DRAWINGS

Further features and advantages of the system and method according to the invention will become apparent from the following description of preferred embodiments, given by way of indicative, non-limiting examples, with reference to the accompanying drawings, in which:

FIG. 1 shows an example of interaction between master device and slave device provided in an embodiment of the method;

FIG. 2 is a block diagram showing some steps included in an embodiment of the method according to the invention;

FIG. 3 is a block diagram showing some steps included in another embodiment of the method according to the invention;

FIG. 4 diagrammatically shows some reference frames and transformations between the reference frames adopted in some embodiments of the method according to the invention;

FIG. 5 shows an example of interaction between master device and slave device included in a method embodiment;

FIG. 6 shows an example of a slave device when in a limited teleoperation state, according to an embodiment of the method;

FIG. 7 diagrammatically shows a robotic system for teleoperated surgery, according to an embodiment of the system of the present invention;

FIG. 8 diagrammatically shows an example of a slave device, according to an embodiment;

FIG. 9 diagrammatically shows some possible steps of the method, according to some embodiments.

### DETAILED DESCRIPTION

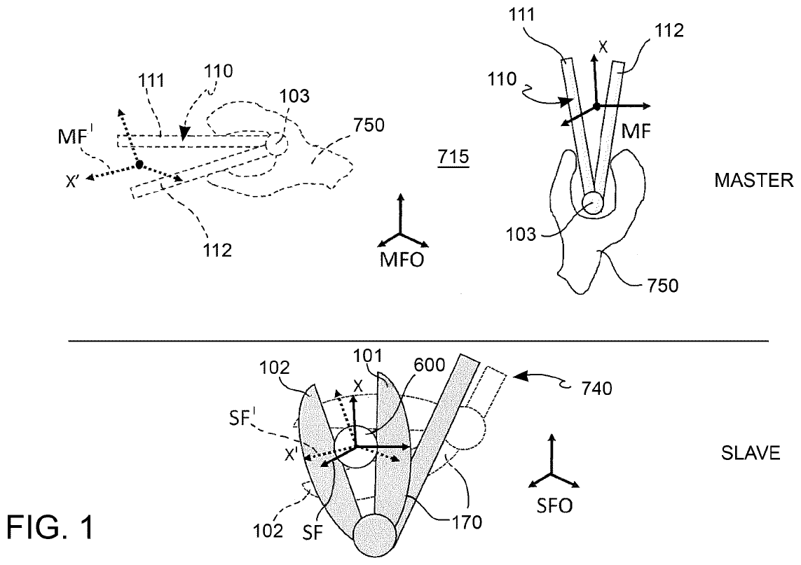

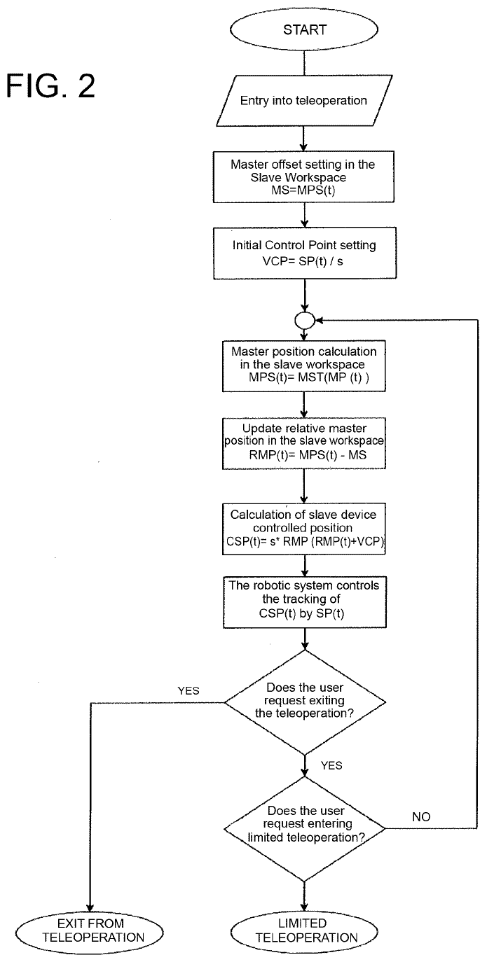

With reference to FIGS. 1-9, a method for controlling a robotic system for medical or surgical teleoperation is described.

Such a robotic system comprises at least one master device, which is hand-held, mechanically unconstrained on the ground (“mechanically ungrounded”) and adapted to be moved (for example, held in the hand) by an operator; and at least one slave device comprising a surgical instrument adapted to be controlled by the master device, so that movements of the slave device, or of the surgical instrument of the slave device, referred to one or more of a plurality of N controllable degrees of freedom are controlled by respective movements of the master device, according to a master-slave control architecture.

The method firstly comprises the steps of defining a first state of the system and a second state of the system.

The first state of the system corresponds to a state of teleoperation with fully enslaved following (tracking), in which the surgical instrument of the slave device (or at least one control point, belonging to or integral with the surgical instrument of the slave device) is enslaved and follows the master device in each of the degrees of freedom of said plurality of N controllable degrees of freedom. For brevity, reference will be made below to said “first state of teleoperation with fully enslaved following” even with the equivalent terminology for the purposes of this description: “first full teleoperation state”.

The second system state corresponds to a limited teleoperation state, in which the surgical instrument of the slave device, or at least the aforesaid control point of the surgical instrument of the slave device, is decoupled from the master device with reference to at least one decoupled degree of freedom, and is enslaved to the master device only in a subset of said plurality of N controllable degrees of freedom which excludes said at least one decoupled degree of freedom.

The method then includes providing, in the aforesaid robotic system, a control means for controlling system state transitions; and for controlling transitions between the aforesaid first state of the system and second state of the system, by the operator, by actuating the aforesaid control means for controlling state transitions.

The plurality of controllable degrees of freedom comprises degrees of freedom of translation and degrees of freedom of orientation.

The aforesaid second limited teleoperation state is a state of repositioning of the master device, in which the aforesaid at least one decoupled degree of freedom comprises all the degrees of freedom of translation. Thus, the surgical instrument of the slave device, or the at least one control point of the surgical instrument of the slave device, does not follow the master device in translation.

As noted above, the second state of the system allows the operator to lock the movement of some degrees of freedom of the slave device, and more precisely of some degrees of freedom of the surgical instrument of the slave device, while allowing the control of the remaining degrees of freedom of the surgical instrument of the slave device by means of the master device.

According to an implementation option, the enslaved movements and the degrees of freedom refer to a control point, belonging to or integral with the surgical instrument of the slave device. For example, such a control point can be a point of the surgical instrument with locked translations, while other points of the slave kinematic chain, such as points being integral with motorized micro-manipulators and/or associated links in rotational joints, can translate also during the aforesaid second limited teleoperation state.

It should be noted that in an implementation option, consistent with the aforesaid definition of control point, any translations due to the rotation dynamics of the human wrist and/or the rotation dynamics of the slave surgical instrument are allowed to be transmitted to the slave device for reasons of usability. Therefore, in such a case, the statement “during the second limited teleoperation state the control point does not follow the master device in translation” means, preferably, that any translations due to the rotation dynamics of the human wrist and/or to the rotation dynamics of the slave surgical instrument are in any case transmitted to the slave device for reasons of usability.

In accordance with an embodiment of the method, the plurality of controllable degrees of freedom comprises degrees of freedom of translation and degrees of freedom of orientation.

According to an embodiment of the method, the aforesaid subset of controllable degrees of freedom comprises at least two degrees of freedom of orientation, and thus said surgical instrument of the slave device, or the at least one control point of the surgical instrument of the slave device, follows the master device in the aforesaid at least two degrees of freedom of orientation.

In an implementation option, the second limited teleoperation state provides that the surgical instrument of the slave device follows the master device in at least two degrees of freedom of orientation, or two degrees of freedom of rotation, and does not follow the master device in any degree of freedom of translation.

Thereby, the surgical instrument of the slave device is decoupled from the point of view of the degrees of freedom of translation from the unconstrained master device. For example, in the second limited teleoperation state, only the degrees of freedom of pitch and yaw of the control point of the surgical instrument of the slave device are enslaved. In this implementation option, there can also be degrees of freedom of rotation, for example of roll which are not enslaved when in the second limited teleoperation state.

In an implementation option, the second limited teleoperation state provides that the control point of the surgical instrument of the slave device follows the master device in all the degrees of freedom of rotation (for example the aforesaid degrees of freedom pitch, yaw and roll) and does not follow the master device in any degree of freedom of translation.

According to an embodiment of the method, the aforesaid plurality of controllable degrees of freedom further comprises at least one open/close (i.e., opening/closing) degree of freedom (hereinafter also referred to by the widely adopted term “grip”).

In an implementation option, the second limited teleoperation state provides that the surgical instrument of the slave device, or said at least one control point of the slave device, follows the master device in all the degrees of freedom of rotation and in the degree of freedom of open/close (for example, in all the degrees of freedom defined above as pitch, yaw, roll and grip), and does not follow the master device in any degree of freedom of translation.

The controllable degree of freedom of open/close can be controlled by a degree of freedom of opening/closing provided on the body of the unconstrained master device which can be elastic, or by any internal degree of freedom provided in the master device (for example an internal degree of freedom of distance/approach along a rectilinear trajectory; and/or a button) which can be elastic, as well as by means of a control interface, for non-limiting example, comprising a pressure sensor.

According to another implementation option, the method provides that, in the second limited teleoperation state, the surgical instrument of the slave device, or at least the aforesaid control point of the slave device, operates as follows: it follows the master device in all the degrees of freedom of orientation; it does not follow the master device in translation; with reference to the degree of freedom of open/close, it follows the master device only in the opening direction, and does not follow the master device in the closing direction.

According to another implementation option, the method provides that, in the aforesaid second limited teleoperation state, the surgical instrument of the slave device, or the at least one control point of the slave device, operates as follows: it follows the master device in all the degrees of freedom of orientation, it does not follow the master device in translation, and, with reference to the degree of freedom of open/close, it follows the master device only in the closing direction, and does not follow the master device in the opening direction.

Such an implementation option can be useful, for example, to avoid an involuntary opening of the slave surgical instrument, when it is in the second state, so as not to change the gripping condition during the second state.

In accordance with another embodiment of the method, in which the plurality of controllable degrees of freedom further comprises at least one degree of freedom of open/close (i.e., opening/closing), in the aforesaid second state of limited teleoperation, the surgical instrument of the slave device, or the at least one control point of the slave device, follows the master device in all the degrees of freedom of orientation and does not follow the master device in the degree of freedom of open/close and does not follow the master device in translation.

According to an embodiment, during said second limited teleoperation state, the translational degrees of freedom of the slave device 170 are not enslaved to the master device 110 for a limited duration preferably less than the duration of said second limited teleoperation state.

According to an implementation option, the transition step towards the second limited teleoperation state has a long time constant so that during the transition there is slowed translational movement of the slave device and at the same time accumulation of displacement of the master-slave mapping. Upon returning to a first state of fully enslaved teleoperation, such an accumulated offset is applied. According to an implementation option, during the second limited teleoperation state with a long time constant, the degree of freedom of grip (opening/closing G) can always be not enslaved.

According to a preferred implementation option, during the second limited teleoperation state with a long time constant, the translational degrees of freedom of the slave device 170 are not enslaved to the master device 110 for a limited duration which can be much shorter with respect to the overall duration of the mapping and/or the duration of the limited teleoperation state. For example, between the actuation time t0 of the state transition control means towards the second limited teleoperation state and the actuation time t1 of the state transition control means towards the first fully enslaved teleoperation state, the master device 110 travels a space dM and the slave device 170, due to limited dynamics, travels a space dS<dM/s. The overall effect is a repositioning offset Q=dM/s−dS.

Since the dynamics are slowed down as a function of the time t1−t0, the longer the duration of the second state, the greater the accumulated offset. For example, if the total duration of the second limited teleoperation state is 2 seconds (t1−t0=2 s), and the slowing down time constant of the slave device is equal to 4 seconds (so that the slave speed reaches zero in such a time), it follows that in 2 seconds the slave device does not slow down completely and when it returns to a state of fully enslaved teleoperation, its movement resumes at full speed, having however also accumulated offsets for the mapping.

In accordance with an embodiment of the method, the aforesaid first state of the system corresponds to an operating state in which the slave device acts during a surgical operation; and the aforesaid second state of the system corresponds to a preparation and/or accommodation and/or repositioning state of the unconstrained master device in a workspace thereof.

Furthermore, the aforesaid transitions are adapted to allow establishing a desired relationship, determined by the operator during the second system state, between a master device workspace, corresponding to the workspace in which the control movement of the master device is defined in the second system state, and the slave device workspace, in which the corresponding movement of the surgical instrument of the slave device, or of the control point of the surgical instrument, is defined.

In accordance with an embodiment, the robotic system is controlled so as to achieve a predeterminable repositioning condition, in which a predetermined relative repositioning between said master device workspace and said slave device workspace is provided for.

According to an implementation option, the predeterminable repositioning condition reached is maintained.

According to an implementation option, the aforesaid predeterminable repositioning condition provides that the mapping center is positioned at the current position of the slave device (170), for example at the aforesaid control point (600).

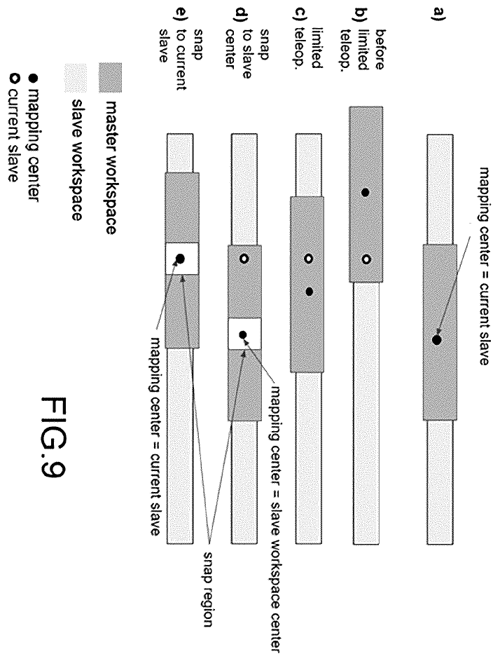

With reference to the aforesaid embodiment, consider FIG. 9 which diagrammatically shows some possible steps of the method, according to some embodiments, and in particular a) a teleoperation start condition, b) a condition before entering a limited teleoperation state, c), d) e) some possible conditions in a limited teleoperation state, according to some possible embodiments.

As mentioned above, in various operating conditions, it can be convenient for the user to control the entry into said second limited teleoperation state (FIG. 9-b), so as to obtain a relative repositioning between the master workspace 715 and the slave workspace (FIG. 9-c).

According to an implementation option, the method includes reaching a predeterminable repositioning condition. Once the predeterminable repositioning condition has been reached, the system can maintain said condition reached by snapping thereto.

A signal of reaching and/or snapping said predeterminable repositioning condition can be provided.

The system can recognize having reached a repositioning condition and automatically lock it until it returns to a first state of fully enslaved teleoperation. According to an implementation option, as long as the master device is near such a point (“snap region”) the mapping between master device and slave device is not modified, and moving away from such a region the normal mapping is applied.

According to an implementation option, a predeterminable repositioning condition provides that the mapping center of the master workspace 715 coincides with the center of the slave workspace (“snap to slave center”, FIG. 9-d).

According to an implementation option, a predeterminable repositioning condition provides that the mapping center of the master workspace 715 coincides with the current position of the slave device (“snap to current slave” FIG. 9-e), for example coinciding with the control point 600 of the slave device 170.

For example, the relative repositioning between master device and slave device is constrained to the value zero when the proposed offset is within a range specified by a threshold; when such a condition is obtained, the operator receives an audible feedback and thus understands to have centered the master workspace within the slave workspace. For example, considering the situation in slave coordinates, an offset tolerance of 1 cm can be used (10 cm in master space due to a scaling factor of 10×according to this hypothesis) within which the repositioning obtained always gives the value zero.

According to another implementation option, the described approach can be applied to the center that is common for several slaves. This may have no impact on the movement of the slave device in transition or on the final movement because it acts only on the offset.

According to different implementation options of the method, given N controllable degrees of freedom, the method comprises managing any subset of the N controllable degrees of freedom as decoupled degrees of freedom, comprising any number of decoupled degrees of freedom between 1 and N−1.

Some implementation options related to the degree of freedom of “grip” or “open/close” are disclosed below.

In an implementation option, in the second limited teleoperation state the degree of freedom of grip (opening/closing G) of the slave device 170 is completely enslaved to the master device 110. Thereby, the user is allowed to release the gripping condition during the repositioning of the master device in the master workspace 715 so as to avoid generating potential damage, for example to tissues.

In another implementation option, in the second limited teleoperation state the degree of freedom of grip (opening/closing G) of the slave device 170 is not enslaved by the master device 110. Thereby, during the repositioning of the master device in the master workspace 715, the user is allowed to reliably maintain a satisfactory gripping force for example on a surgical needle not subject to modulation of the gripping force.

In another implementation option, in the second limited teleoperation state the degree of freedom of grip (opening/closing G) of the slave device 170 is enslaved to the master device 110 only in the closing direction, and is not enslaved to the master device in the opening direction. Thereby it is possible to tighten and/or maintain the grip in the second limited teleoperation state. For example, a sensor capable of estimating the gripping force can be associated with the slave device, either directly, for example by placing it inside the grip tweezers 101, 102 of the slave surgical instrument 170, or indirectly, for example by placing it in the robotic manipulator 740 to which the surgical instrument can be associated in a detachable manner, for example on one or more actuators of the robotic manipulator which control the movement of the degree of freedom of opening/closing G of the slave surgical instrument.

As already observed, in an implementation option, the aforesaid second limited teleoperation state is a state of repositioning of the master device, in which the slave device follows the master device only in orientation and not in translation, thus resulting decoupled from the master device from the point of view of translation movements. Thereby, it is possible to maintain the alignment of the orientations between master device and slave device during the first state, during the second state, and during the transitions between the first and second state. Therefore, the transitions between first and second state do not require a dedicated master-slave alignment step.

In the context of hand-held master devices, mechanically unconstrained and capable of being moved while held in the hand by an operator which control enslaved devices according to a scaling, the transition between the first state and the second state can be frequent in response to the need to reposition the master device within the workspace thereof.

According to an implementation option, the plurality of controllable degrees of freedom comprises three degrees of freedom of translation and three degrees of freedom of orientation (roll, pitch, yaw, already disclosed above). A further controllable degree of freedom for opening/closing (“grip”, already described above) can be included.

In accordance with an embodiment of the method (which has already been mentioned previously, and which is described here in more detail), a control point of the surgical instrument comprised in the slave device is defined, and, in the second limited teleoperation state, the translation of the aforesaid control point is inhibited, while the possibility of rotation of the control point is maintained, to vary the orientation of the surgical instrument of the slave device depending on the orientation of the master device, until an alignment condition is reached, in which they have the same orientation (within the limits of a predefined tolerance) between the master device and the surgical instrument of the slave device, while the position of the aforesaid control point, in the reference space of the slave device, remains unchanged.

According to a preferred implementation option, the orientation of the control point can be such as to model the orientation of the slave surgical instrument and preferably of the distal portion of the surgical instrument having at least one free end.

In such a case, the method provides that the operator can control, during the second limited teleoperation state, only the orientation, and optionally also a further degree of freedom of the surgical instrument of the slave device identifiable with the grip of the surgical instrument (opening/closing or “grip”), keeping the position of the control point of the slave surgical instrument fixed. Thereby, the transition between the first and second state, or vice versa, does not affect the alignment between the master device and the surgical instrument of the slave device.

According to an implementation option of the aforesaid embodiment, the surgical instrument of the slave device comprises a distal joint for the connection with the slave device and two tips configured to grip and guide a surgical needle, and the aforesaid control point of the surgical instrument corresponds to a physical point placed between said distal joint and the end of the tips. Each tip (or nozzle) can comprise a rigid body and have a free end. The tips of the surgical instrument are not necessarily intended to grasp a surgical needle, although they may be.

According to an implementation option, the control point is a midpoint between the tips of the surgical instrument of the slave device.

In another implementation option, the control point of the surgical instrument corresponds to the point where the tips (nozzles) grasp the surgical needle, when closed in the gripping configuration.

According to other implementation options, the aforesaid control point corresponds to a real point which is integral with the physical point illustrated above, or a virtual point fixedly correlated with the physical point illustrated above.

In accordance with an implementation option of the method, at the end of a transition from the second state to the first state, the master device and the surgical instrument of the slave device are aligned, i.e., they have the same orientation, because the master device and the control point are aligned.

Thereby, when exiting the limited teleoperation step, the master device and the surgical instrument of the slave device are already aligned, because the master device and the control point are aligned.

According to an embodiment of the method, before entering into the first system state, of teleoperation with fully enslaved tracking, a zero point is defined, which correlates the master device workspace and the slave device workspace, for translation.

In such a case, at an exit from the second state, at the end of the limited teleoperation step, the resulting translation offset between the master device and control point of the surgical instrument of the slave device is stored and added to a current zero point, so that, in the subsequent teleoperation step with fully enslaved tracking, the control of the slave device by the master device obeys a relationship which takes into account said translation offset which occurred during the limited teleoperation step.

In other words, the zero point is reassigned when exiting the limited teleoperation step.

According to an implementation option, during the second limited teleoperation state the calculated offset is limited to a maximum value which cannot be exceeded by the operator.

According to an implementation option, at the end of a transition from the second state to the first state, and vice versa, the master device and the surgical instrument of the slave device are aligned, i.e., they have the same orientation because the control point of the surgical instrument and the master device are aligned.

According to an implementation option, during the aforesaid transitions between the first state and the second state, the kinematic parameters of speed and accelerations are limited so as to make regular the locking or unlocking of the degrees of freedom.

With reference to the kinematic parameters, without losing generality, let's assume the use of a control algorithm, which has as its primary objective the tracking (following) of the master device by the surgical instrument of the slave device, or the control point of the surgical instrument according to the coupled degrees of freedom. Kinematic parameters such as maximum speeds and accelerations expressed in the workspace of the slave device, or with respect to the joints of the actuation system of the slave device, are then considered as constraints of said objective.

Thus consider a control algorithm which iteratively performs an optimization of the enslavement first with respect to the primary target and then constrains the result with respect to the indicated constraints, or a control algorithm which performs such an optimization taking such constraints into account, by means of some restriction of the of search of the result.

In the transition between full teleoperation and limited teleoperation, the contribution to translational tracking, expressed in the workspace of the slave device, will be canceled.

It should be noted how the decoupling of the translational component can be expressed as a translational speed constraint expressed in the workspace of the slave device.

Such parameters describing the speed and acceleration limits are considered constant at the beginning and at the end of the transition. In this sense, the adaptation of such parameters can be varied continuously during the transition according to a suitable interpolation formula. Given the initial state A and the final state B and a transition time T and transition start time to, given a parameter P such as the speed limit along a given actuation axis, then it is possible to use the linear interpolation formula of this constraint:

P(t)=P_B(t−t0)/T+P_A(1−(t−t0)/T)

It should further be considered that for some parameters a solution which interpolates the value quadratically is rather preferable. Therefore, let's introduce a variable A as a function of time t and the general function:

λ(t)=(t−t0)/T

P(λ)=q3λ³+q2λ²+q1 λ+q0

where a, b, c can be calculated so that P(0)=P_A, P(1)=P_B. It should be noted that such a formulation comprises the formulation of linear interpolation for an appropriate choice of parameters.

According to an implementation option, such kinematic parameters are limited differently depending on whether the transition is a state transition towards the second state of limited teleoperation or is a state transition towards the first state.

In accordance with an embodiment, in the transitions involving the limited teleoperation state, the trajectories of the limited degrees of freedom do not have appreciable discontinuities in the main kinematic parameters. In particular, speeds and accelerations are limited, while entering and exiting from the limited teleoperation state, so as to soften the freezing of the degree of freedom concerned and then resume the actuation thereof without generating distortions or jolts perceptible by the operator.

The criteria used for limiting the kinematic parameters during state changes involving limited teleoperation are preferably more stringent than those used in the full teleoperation state. Such criteria are preferably to be understood as different in the case of entry and exit from the limited teleoperation step. During the limited teleoperation step, the not-limited degrees of freedom can be subject to a more stringent limitation of the kinematic speed and acceleration parameters than the limitation active during full teleoperation.

Some further details will be provided below on the reference frames used to coordinate the movement of the master and slave devices, according to a particular implementation example of the method.

Due to possible limitations in the workspace of the surgical instrument of the slave device, with respect to the workspace of the unconstrained master device, the surgeon must be as empowered as possible to adjust his posture and position as well as the placement of the hand(s) holding the unconstrained master device within the master device workspace in a manner which facilitates the surgical or microsurgical procedure.

According to the implementation example presented below, the limited teleoperation step or state is perceived by the operator with the mechanical analogue of the “clutch”, which temporarily decouples the translations of the master device from those controlled by the slave device. The existence of a geometric object referred to as a “control point” allows considering and therefore managing the translational component of the teleoperation not as a static 1-1 mapping between the master space and the slave space, but as a constrained variation of translational speeds between the master and slave device during the states where such translational mapping is allowed, i.e., during said first full teleoperation state.

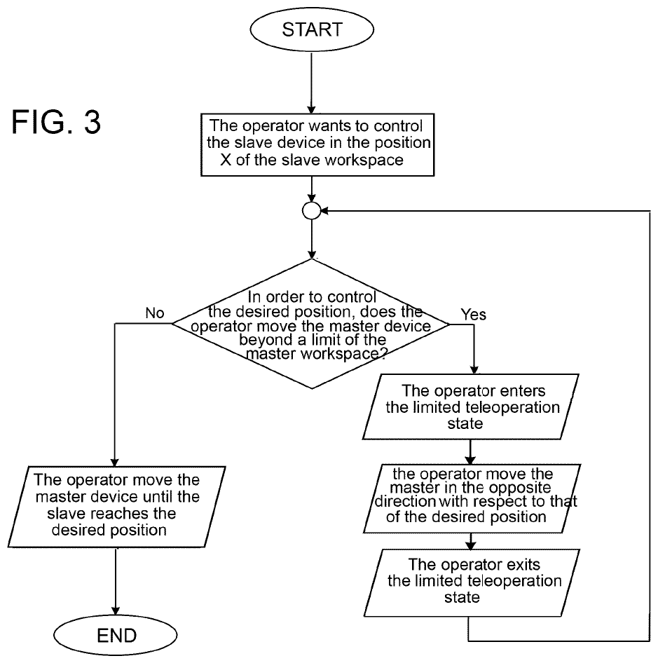

In particular, this is useful if the operator needs to perform relatively large displacements of the slave device which are not allowed into/from the workspace of the master device due to scaling relationship (i.e., scale ratio or simply “scaling”). In this scenario, the operator can reach any position belonging to the slave workspace using the algorithm presented in the flowchart in FIG. 3 (without losing generality, only one degree of freedom was considered).

According to an embodiment, the aforesaid control means for controlling state transitions comprise a control button.

According to an implementation option, such a control button is a control pedal.

According to an implementation option, the control button is integral with the unconstrained master device, for example it is placed on the body of the master device.

In an implementation option, entering and staying in a limited teleoperation state is associated with pressing a control pedal. Releasing the control pedal automatically causes the transition from the second limited teleoperation state to the first full teleoperation state.

In an implementation option, the fact of being in teleoperation is a sufficient condition so that pressing the control pedal determines entering the limited teleoperation state.

An implementation option of the limited teleoperation method is shown below.

To this end, a Virtual Control Point (VCP) can be defined, which is used as the origin of the reference coordinate frame for the mapping between the pose of the master device in the master workspace and the pose of the surgical instrument of the slave device (for brevity also referred to only as “slave device”) in the workspace of the slave surgical instrument.

In an implementation option, two reference coordinate frames are defined for each of the master and slave devices (in the discussion the terms “coordinate frame” and “reference frame” will be used as equivalents): a “Master Frame Origin” (MFO) coordinate frame, for example integral with the tracking system, and a “Master Frame” (MF) coordinate frame, for example integral with the unconstrained master device, used to describe the pose (comprising the set of information “MP position, orientation”) of the unconstrained master device; and a “Slave Frame Origin” (SFO) coordinate frame and a “Slave Frame” (SF) coordinate frame used to describe the pose (including the set of information “MP position, orientation”) of the slave device. “Slave device” indicates the control point of the surgical instrument of the slave device.

The position of the master device MP is defined as the relative position (translational component) of the “Master Frame” MF coordinate frame with respect to the “Master Frame Origin” MFO coordinate frame, and the position of the slave device SP is defined as the relative position of the “Slave Frame” SF coordinate frame with respect to the “Slave Frame Origin” SFO coordinate frame which is integral with the slave robotic system.

In an implementation example, the movements of the master and slave devices are constrained in separate and independent workspaces, which respectively limit the position of the master device MP and the position of the slave device SP.

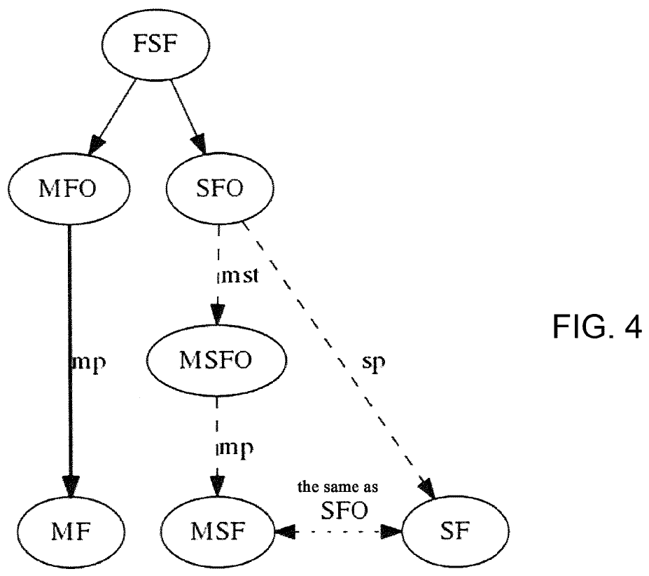

In the embodiment diagrammatically shown in FIG. 4, a Fixed Reference System (FSF) is considered. It is thus possible to express the “Master Frame Origin” MFO and “Slave Frame Origin” SFO coordinate frames in such a fixed reference system FSF and define the “master slave transformation” (MST) which allows mapping transformations related to “Master Frame Origin” MFO in transformations related to “Slave Frame Origin” SFO.

Therefore, it is possible to define the “Master frame in slave frame origin” (MSFO) coordinate frame in the “Slave Frame Origin” SFO reference frame, obtained by applying the transformation MST to the reference frame SFO, and further to define the “Master in Slave Frame” (MSF) coordinate frame obtained by applying the transformation from “Master frame” MF to “Master frame origin” MFO with respect to MSFO.

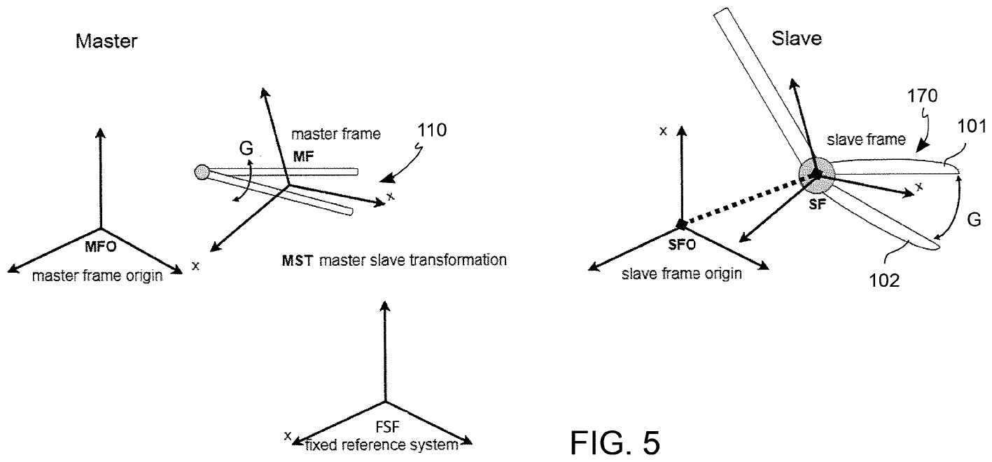

The reference frames mentioned below are shown in particular in FIG. 5:
“master frame” (MF), or “master reference frame”;
“master frame origin” (MFO), or “master reference frame origin”;
“slave frame” (SF), or “slave reference frame”;
“slave frame origin” (SFO), or “slave reference frame origin”;
“fixed reference system” (FRS), or fixed external reference system;
“master to slave transformation” (MST);
“master frame in slave frame origin” (MSFO), or “master reference frame in the slave reference frame origin”.

Considering a situation of surgical application in which the ratio between movements of the master and slave devices is 1:1 (application situation in which the method described in this disclosure can be used), and a full teleoperation condition, the movements of the surgical instrument (hereinafter also referred to as the “end-effector”) of the slave device are controlled by the movements of the unconstrained master device, to which the slave device is enslaved.

This is equivalent, leaving out any translational offsets as explained below, to controlling the movement of the slave device, represented by “Slave Frame” SF, using the “Master frame in slave frame” MSF as a reference. In particular, from the translational point of view, the teleoperation can be seen as following, by the slave device position SP, the coordinate frame MPS, where MPS is the position of the “Master frame in slave frame” MSF in the “Slave Frame Origin” SFO reference frame.

Upon entering a teleoperation state, also due to the mechanically unconstrained nature of the master device(s), there is no positional relationship between the position of the master device in the slave space MPS and the current position of the slave device SP.

It is thus necessary, when entering the teleoperation state, to subtract an offset MS from the position MPS: MPS(0) so that, assuming that at the entry of the teleoperation the slave is at the origin of the reference frame thereof, the resulting position coincides at the initial instant with the position of the slave device SP. Thereby, such a slave device will be controlled only by movements of the master device which occurred after entering the teleoperation.

Such a position in the space of the slave device is referred to as “Relative Master Position” (RMP), and is calculated in each instant of teleoperation as:

RMP: RMP(t)=MPS(t)−MS.

In a further application context, in which the method described in the present disclosure can be carried out, i.e., if the robotic teleoperation system is configured to perform microsurgery operations, it is necessary to consider a scale factor s (with s<1), describing a desired/required relationship between the range of motion of the slave device and the range of motion of the master device. For this reason, in this case it is necessary to define a scaled master position CSP defined as:

CSP=s·RMP.

Taking into account the scale factor, the volume that the scaled master position CSP can accomplish is equal to s³ times the workspace of the master device. For this reason, as the scaling increases (and therefore the scale factor s decreases), only a small portion of the slave workspace is reachable during the teleoperation.

To overcome this drawback, as well as the fact that the slave device may not be in the reference frame origin thereof during the entry into teleoperation, an implementation option defines the aforementioned “Virtual Control Point” (VCP) which is used as the origin of the reference coordinates for the mapping mechanism between the pose (position and orientation) of the master device and the pose (position and orientation) of the slave device.

The insertion of this point, which can be controlled by the user, allows redefining the position commanded to the slave device, in this implementation option, as:

CSP: CSP=s·(RMP+VCP).

The point VCP is initialized at each t=0 in which a teleoperation is started respecting the following formulation:

VCP=SP(0)/s.

Using any scale factor s, the operator is thus always able to reach any point of the workspace of the slave device, regardless of the size thereof, by virtue of the control of the “Virtual Control Point” VCP parameter.

In an implementation option, the method which indirectly allows the operator to intuitively control the “Virtual Control Point VCP” parameter is the use of a new teleoperation state, corresponding to the second limited teleoperation state. In this second limited teleoperation state, the slave device follows the master device only in orientation and not in translation.

During the limited teleoperation state, according to an implementation option, a temporary virtual control point TVCP is defined as:

TVCP(t)=VCP+RMP(t_0)−RMP(t)==VCP+MPS(t_0)−MPS(t),

where MPS(t) is the current position of the master in the slave space, and MPS(t_0) is the position of the master in the slave space at the instant of entry into limited teleoperation.

When exiting the limited teleoperation state, then the temporary virtual control point TVCP is assigned to the virtual control point: VCP:=TVCP.

As those skilled in the art will appreciate, upon returning to a full teleoperation state, the new definition of the virtual control point VCP does not involve a change in the position commanded to the slave device CSP. In other words, the dynamic redefinition of the “Virtual Control Point” VCP described above allows the fact that no controlled translation of the slave device occurs at the entry of the limited teleoperation state, or during and at the exit of the limited teleoperation state.

An application example of the “limited teleoperation” state is now described, according to the implementation options described above.

Without losing generality, the example will contemplate a teleoperation having a single translational degree of freedom, denoted by “x”.

Again without losing generality, it is assumed that the mapping from the space of the master device to the space of the slave device is equal to the identity, or MPS=MP.

In this context, suppose at an instant of time t=0 the entry into teleoperation with the master device with MP(0)=10 cm and SP(0)=2 cm in the respective reference frames.

Also suppose the master space has a maximum excursion of +−30 cm, and the slave space has a maximum excursion of +−7 cm.

Further suppose being in the teleoperation state with a scale factor of 10×, or s=0.1 (1 cm of displacement of the master device equals 1 mm of displacement of the slave device).

At the instant of entry into teleoperation, the offset MS is fixed: MPS(0)=10 cm and the control point VCP=2 cm/0.1=20 cm.

Thereby, the controlled position at the time instant t turns out to be

CSP(t)=s·(RMP(t)+VCP)=0.1·(MPS(t)−10+20).

It should be noted that in t=0, MPS(0)=10 cm and CSP(0)=0.2 mm and there is no translation when entering a full teleoperation state.

Again merely by way of example, now assume that the operator must reach the point in the space of the slave device x=4.5 cm, belonging to the workspace of the slave device. It should be noted that, without exiting the full teleoperation state, using the entire workspace of the master device it would be possible to control, at most, the position

CSP=0.1·(max(MPS)−10+20)=0.1·(30−10+20)=4 cm.

To reach such a position, the operator at time t_0 can perform, for example, the following actions:
1) Entry into the limited teleoperation state. The temporary control point TVCP is set to TVCP(t)=VCP+MPS(t_0)−MPS(t)=20+10−MPS(t)
2) The operator moves the master device until it reaches the position x=0. The temporary control point TVCP then assumes the value

TVCP=20+10−0=30 cm
3) The operator exits the limited teleoperation state and re-enters the full teleoperation state. The parameter VCP is updated with the current TVCP value, or 30 cm. It should be noted that the new controlled position CSP is the same as that before entering into limited teleoperation:

$$\begin{matrix}CSP ( t ) = s \cdot ( MPS ( t ) - MS + VCP ( t ) ) = \\ = 0.1 \cdot ( 0 - 10 + 30 ) 2 cm\end{matrix}$$

4) The operator moves the master device until it reaches the position x=25 cm, belonging to the master workspace. Thereby the new controlled position is equal to

$$\begin{matrix}CSP ( t ) = s \cdot ( MPS ( t ) - MS + VCP ( t ) ) = \\ = 0.1 \cdot ( 25 - 10 + 30 ) = 4.5 cm\end{matrix}$$

According to a further implementation option of the method, the change of state between teleoperation and limited teleoperation can occur in a non-punctual finite time, in which the speed and acceleration are progressively limited, allowing the slave device to stop without introducing excessive motion distortions.

Similarly, according to another implementation option, the change of state between limited teleoperation and teleoperation can occur in a non-punctual finite time in which the initial speeds and accelerations commanded to the slave device are limited to avoid excessive motion distortions.

According to another implementation option, the limited teleoperation step temporarily limits/prevents the teleoperation of the degree of freedom of opening/closing (“grip”) of the instrument, or at the moment of entry into the limited teleoperation step the degree of opening/closing (“grip”) of the surgical instrument of the slave device is frozen for the entire stay in the aforesaid second limited teleoperation state.

According to another implementation option, the limited teleoperation step prevents only a subset of the possible states related to a given degree of freedom.

According to another implementation option, the limited teleoperation step prevents the closure and allows the opening allowing the surgical instrument of the slave device. In other words, according to this implementation option, the degree of freedom of opening/closing is only partially locked in the second limited teleoperation state, since control is allowed in opening but not in closing.

According to an implementation option of the method, in the transitions involving the limited teleoperation state the trajectories of the limited degrees of freedom do not have appreciable discontinuities in the main kinematic parameters. In particular, speeds and accelerations are limited, in entry and exit from the limited teleoperation state, so as to soften the freezing of the degree of freedom concerned and then resume the actuation thereof without generating distortions or jolts perceptible by the user.

According to another implementation option of the method, the criteria used for limiting the kinematic parameters, during state changes involving the limited teleoperation, provide that the acceleration and/or deceleration of the slave in the entry or exit step of limited teleoperation are much lower than the maximum acceleration value in the aforesaid first full teleoperation state, and more stringent than those used by full teleoperation. Such criteria, in a typical implementation option, are different in the case of entry and exit from the limited teleoperation step.

According to another implementation option of the method, the implementation of the control means for controlling the system state transitions modifies the master-slave control parameters, such as accelerations and translation speeds, until the entry transition into limited teleoperation or exit transition from limited teleoperation has been completed.

In an implementation option, during the limited teleoperation step, the non-limited degrees of freedom can be subject to a more stringent limitation of the kinematic speed and acceleration parameters than the limitation active during full teleoperation.

A robotic system for teleoperated surgery is now described, adapted to be controlled by the aforesaid method for controlling a robotic system.

Such a system comprises at least one master device, which is hand-held, mechanically unconstrained and adapted to be moved without mechanical constraints by an operator, so that the master device can move freely inside a predefined workspace thereof. The system further comprises at least one slave device comprising a surgical instrument adapted to be controlled by the master device, so that movements of the slave device (or movements of at least one control point, belonging to or integral with the surgical instrument of the slave device), referred to one or more of a plurality of N controllable degrees of freedom, are controlled by respective movements of the master device, according to a master-slave control architecture.

The system further comprises a control means for controlling system state transitions, actuatable by the operator; and a control unit, operatively connected to both the master device and to the slave device, and to the control means for controlling state transitions.

The control unit is configured to control the system so as to perform a robotic system control method according to any of the previously disclosed embodiments.

According to an embodiment, the robotic system control unit is configured to control the robotic system so as to perform the following actions:
defining a first state of the robotic system, corresponding to a state of teleoperation with fully enslaved following, in which the surgical instrument 170 of the slave device 740, or at least a control point 600, belonging to or integral with the surgical instrument 170 of the slave device 740, is enslaved and follows the master device 110 in each of the degrees of freedom of the aforesaid plurality of N controllable degrees of freedom;
defining a second state of the robotic system, corresponding to a limited teleoperation state, in which the surgical instrument 170 of the slave device 740, or at least the aforesaid control point 600 of the surgical instrument 170 of the slave device 740, is decoupled from the master device 110 with reference to at least one decoupled degree of freedom, and is enslaved to the master device only in a subset of the plurality of N controllable degrees of freedom which excludes the aforesaid at least one decoupled degree of freedom;
providing, in the robotic system, control means for controlling system state transitions;
controlling transitions between the aforesaid first state and second state of the robotic system, by the operator, by actuating the means for controlling system state transitions.

The plurality of controllable degrees of freedom comprises degrees of freedom of translation and degrees of freedom of orientation.

The aforesaid second limited teleoperation state is a state of repositioning of the master device 110, in which the aforesaid at least one decoupled degree of freedom comprises all the degrees of freedom of translation, and therefore the surgical instrument 170 of the slave device 740, or the aforesaid at least one control point 600 of the surgical instrument 170 of the slave device 740, does not follow the master device in translation.

According to an embodiment of the system, the aforesaid control means for controlling state transitions comprise a control button, or a control pedal, which can be pressed and/or kept pressed and/or released by the operator. In such a case, the second limited teleoperation state is activated, during teleoperation, by keeping the control pedal pressed, and is deactivated by releasing the control pedal.

According to an embodiment, the system is a robotic system for teleoperated microsurgery, and the aforesaid surgical instrument of the slave device is a microsurgical instrument.

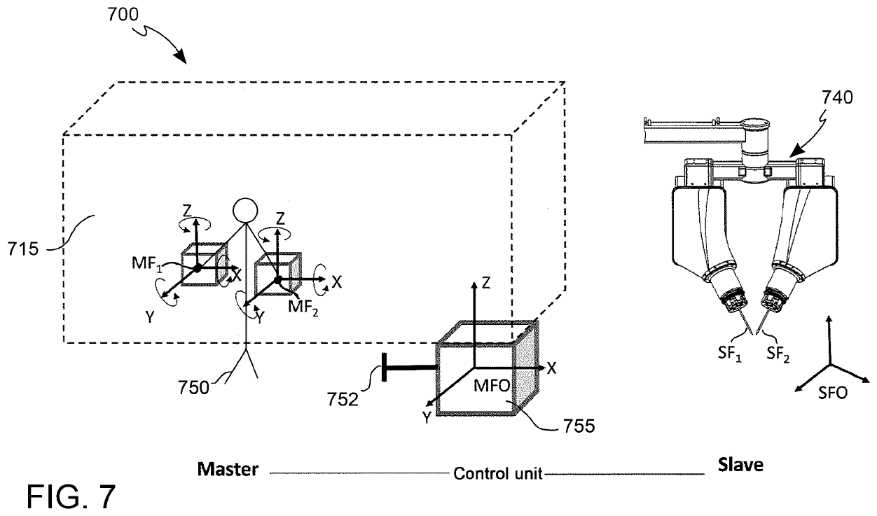

In the embodiment shown in FIG. 7, a robotic system 700 for teleoperated surgery is illustrated, comprising at least one hand-held master device, mechanically unconstrained and adapted to be moved by an operator 750 (in the example shown, two unconstrained master devices are shown MF1, MF2 inside a workspace 715) and at least one slave device 740 comprising a surgical instrument adapted to be controlled by the master device (in the example shown, two surgical instruments SF1, SF2 of the slave device 740 controlled by respective master devices MF1, MF2 are shown).

The robotic system 700 shown in FIG. 7 further comprises a control means for controlling system state transitions 752, actuatable by the operator 750, and a control unit, operatively connected to both the unconstrained master device and to the slave device 740, and to the control means for controlling state transitions 752. In the example shown, the control unit is shown as forming part of a console 755 integral with the master workspace 715.

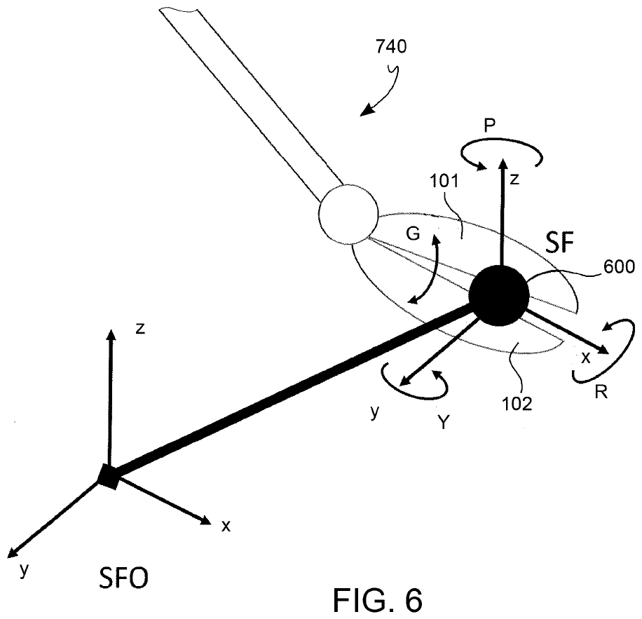

FIG. 6 diagrammatically shows an example of the slave device 740 during the second limited teleoperation state, in which the translations of the control point 600 are locked with respect to the “Slave frame origin” SFO reference frame; in such an example the degrees of freedom of the control point 600 are diagrammatically shown, which remain enslaved during the second state: pitch P, yaw Y and, as required by some implementation options, roll R and/or grip G, i.e., opening and/or closing G of the tips (nozzles) 101, 102 of the surgical instrument SF of the slave device.

FIG. 1 diagrammatically shows an example of the second limited teleoperation state, in which the translations of the control point 600 are locked with respect to the “Slave frame origin” SFO reference frame, even though the master device 110 has performed translation and rotation movements from the pose MF (dotted line) to the pose MF with respect to the “Master frame origin” MFO reference frame in the workspace 715, while held in hand by the operator 750; the control point 600 is shown in this example between the tips (nozzles) 101, 102 of the surgical instrument 170 of the slave device 740.

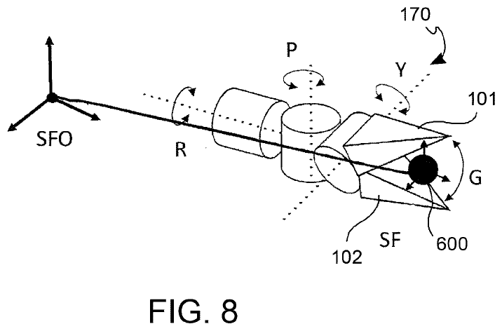

In the example shown in FIG. 8, the surgical instrument 170 of the slave device comprises an articulated wrist having joints (or articulated joints) for actuating roll R, pitch P and yaw Y, in which a control point 600 is defined having translations locked in the reference frame SFO when in the second limited teleoperation state.

FIG. 5 diagrammatically shows a master device 110 having an internal degree of freedom of open/close G′ adapted to control an enslavable degree of freedom of open/close G of the tips 101, 102 of the slave surgical instrument 170. In the example shown, the degree of freedom of open/close G′ of the master device 110 is formed by two rigid parts 111, 112 constrained in a rotational joint 103 to rotate relatively about a common axis.

As can be seen, the objects of the present invention as previously indicated are fully achieved by the method described above by virtue of the features disclosed above in detail.

In fact, the method and the system described above allow performing state transitions, which on the one hand ensure the absolute safety of the patient and the comfort of the surgeon's action, and on the other hand allow effectively maintaining the master-slave alignment, during the state transitions of the teleoperation.

This is obtained by virtue of the features of the method and system described, in particular by virtue of the definition of two different teleoperation states, one of teleoperation with complete enslavement and the other of limited teleoperation (in which the slave device is decoupled from the master device with reference to the degrees of freedom of translation, and is enslaved to the master device only in a subset of the plurality of N controllable degrees of freedom), and by virtue of the possibility offered to the operator (for example, surgeon) to freely control every transition between the aforesaid two states.

Furthermore, as mentioned above, according to a preferred implementation option, in the second limited teleoperation state the orientation of the slave is constantly kept aligned with the orientation of the master by virtue of the fact that the slave device 170, i.e., the control point 600, follows the master device 110 in orientation (and does not follow it in translation), so that when it returns to a state of fully enslaved teleoperation the master device and the slave device—i.e., the control point 600 of the slave device—are aligned. This avoids distortions in the master-slave relationship upon re-entry into fully enslaved teleoperation and also avoids the need for a subsequent recovery of misalignment during the fully enslaved teleoperation step. The need to perform an alignment step between master and slave at the end of the limited teleoperation state is also avoided.

During the second limited teleoperation state, the surgeon is able to reposition or rearrange the unconstrained master device within the workspace of the master device, without imposing any translation of the control point of the surgical instrument of the slave device.

By virtue of the proposed solutions, a teleoperated system for surgery and preferably microsurgery, in which a scaling between the translations of the unconstrained master device and the enslaved translations of the slave control point is provided for, and there is no scaling between the rotations of the master device and rotations of the slave control point, allows maintaining the master-slave alignment in any transition between first and second state, and vice versa.

In order to meet contingent needs, those skilled in the art may make changes and adaptations to the embodiments of the method described above or can replace elements with others which are functionally equivalent, without departing from the scope of the following claims. All the features described above as belonging to a possible embodiment may be implemented irrespective of the other embodiments described.

1. A method for controlling a robotic system for medical or surgical teleoperation, wherein said robotic system comprises at least one master device, which is hand-held, mechanically unconstrained and adapted to be moved by an operator, and at least one slave device comprising a surgical instrument adapted to be controlled by the master device, so that movements of the slave device, or of the surgical instrument of the slave device, referred to one or more of a plurality of N controllable degrees of freedom, are controlled by respective movements of the master device, according to a master-slave control architecture,
wherein the method comprises:
defining a first state of the system, corresponding to a state of teleoperation with fully enslaved following, in which the surgical instrument of the slave device, or at least a control point, belonging to or integral with the surgical instrument of the slave device, is enslaved and follows the master device in each of the degrees of freedom of said plurality of N controllable degrees of freedom;
defining a second state of the system, corresponding to a limited teleoperation state, in which the surgical instrument of the slave device, or at least said control point of the surgical instrument of the slave device, is decoupled from the master device with reference to at least one decoupled degree of freedom, and is enslaved to the master device only in a subset of said plurality of N controllable degrees of freedom which excludes said at least one decoupled degree of freedom;
providing, in said robotic system, a controller for controlling system state transitions;
controlling transitions between said first state of the system and second state of the system, by the operator, by actuating said controller for controlling system state transition;
wherein the plurality of controllable degrees of freedom comprises degrees of freedom of translation and degrees of freedom of orientation,
and wherein said second limited teleoperation state is a state of repositioning of the master device, wherein said at least one decoupled degree of freedom comprises all the degrees of freedom of translation, and therefore said surgical instrument of the slave device, or said at least one control point of the surgical instrument of the slave device, does not follow the master device in translation.

2. A method according to claim 1, wherein said subset of controllable degrees of freedom comprises at least two degrees of freedom of orientation, and wherein said surgical instrument of the slave device, or the at least one control point of the surgical instrument of the slave device, thus follows the master device in said at least two degrees of freedom of orientation.

3. A method according to claim 2, wherein said at least two degrees of freedom of orientation, with reference to which the surgical instrument of the slave device, or the at least one control point of the surgical instrument of the slave device, follows the master device, and is enslaved to the master device, comprise a degree of freedom of pitch and a degree of freedom of yaw.

4. A method according to claim 1, wherein:
in said second limited teleoperation state, said surgical instrument of the slave device, or said at least one control point of the surgical instrument of the slave device, follows the master device in all the degrees of freedom of orientation and does not follow the master device in translation,
wherein said degrees of freedom of orientation comprise a degree of freedom of pitch, a degree of freedom of yaw, and a degree of freedom of roll.

5. A method according to claim 1, wherein the plurality of controllable degrees of freedom further comprises at least one degree of freedom of open/close,
and wherein, in said second limited teleoperation state, said surgical instrument of the slave device, or said at least one control point of the slave device, follows the master device in all the degrees of freedom of orientation and in the degree of freedom of open/close and does not follow the master device in translation.

6. A method according to claim 5, wherein, in said second limited teleoperation state, said surgical instrument of the slave device, or said at least one control point of the slave device, operates as follows:
said surgical instrument of the slave device, or said at least one control point of the slave device follows the master device in all the degrees of freedom of orientation, does not follow the master device in translation, and, with reference to the degree of freedom of open/close, follows the master device only in the opening direction, and does not track the master device in the closing direction.

7. A method according to claim 5, wherein, in said second limited teleoperation state, said surgical instrument of the slave device, or said at least one control point of the slave device, operates as follows:
said surgical instrument of the slave device, or said at least one control point of the slave device follows the master device in all the degrees of freedom of orientation, does not follow the master device in translation, and, with reference to the degree of freedom of open/close, follows the master device only in the closing direction, and does not follow the master device in the opening direction.

8. A method according to claim 1, wherein the plurality of controllable degrees of freedom further comprises at least one degree of freedom of open/close,
and wherein, in said second limited teleoperation state, said surgical instrument of the slave device, or said at least one control point of the slave device, follows the master device in all the degrees of freedom of orientation and does not follow the master device in the degree of freedom of open/close and does not follow the master device in translation.

9. A method according to claim 1, wherein:
said first state of the system corresponds to an operating state in which the slave device acts during a surgery;
said second state of the system corresponds to a preparation and/or accommodation and/or repositioning state of the master device in a workspace;
said transitions are adapted to allow establishing a desired relationship, determined by the operator during the second state of the system, between a master device workspace, corresponding to the workspace in which the control movement of the master device is defined in the second state of the system, and the slave device workspace, in which the corresponding movement of the surgical instrument of the slave device, or of the control point of the surgical instrument, is defined.

10. A method according to claim 1, wherein, in the second limited teleoperation state, translation of said control point is inhibited, while a possibility of rotation of said control point is maintained, in order to vary the orientation of the surgical instrument of the slave device depending on the orientation of the master device, maintaining an alignment condition, having a same orientation, between the master device and the surgical instrument of the slave device, while the position of said control point, in a slave device reference space, remains unchanged.

11. A method according to claim 1, wherein the surgical instrument of the slave device comprises a distal joint for connection with the slave device and two tips configured to grip and guide a surgical needle,
and wherein said control point of the surgical instrument corresponds to a point located between said distal joint and an end of the tips.

12. A method according to claim 1, wherein, at an end of a transition from said second state to said first state, the master device and the surgical instrument of the slave device are aligned, and have a same orientation.

13. A method according to claim 1, wherein, at an end of a transition from said first state to said second state, the master device and the surgical instrument of the slave device are aligned, and have a same orientation.

14. A method according to claim 1, wherein during said transitions between said first state and said second state, the kinematic parameters of speed and accelerations are limited to make regular locking/unlocking of the decouplable degrees of freedom.

15. A method according to claim 14, wherein said kinematic parameters are limited differently depending on whether the transition is a state transition towards said second limited teleoperation state or is a state transition towards said first state.

16. A method according to claim 1, wherein, before entering into the first state of the system, of teleoperation with fully enslaved following, a zero point is defined, which correlates a master device reference space and a slave device reference space, for translation,
and wherein, at an exit from the second state, at an end of the limited teleoperation step, the resulting translation offset between the master device and the slave device is stored and added to a current zero point, so that, in a subsequent teleoperation step with fully enslaved following, control of the slave device by the master device obeys a relationship which takes into account said translation offset which occurred during the limited teleoperation step.

17. A robotic system for teleoperated surgery adapted to be controlled by said robotic system control method, wherein the system comprises:
at least one master device, which is hand-held, mechanically unconstrained and adapted to be moved without mechanical constraints by an operator, so that the master device can move freely inside a predefined workspace;
at least one slave device comprising a surgical instrument adapted to be controlled by the master device, so that movements of the slave device, or movements of at least one control point, belonging to or integral with the surgical instrument of the slave device, said movements being referred to one or more of a plurality of N controllable degrees of freedom, are controlled by respective movements of the master device, according to a master-slave control architecture;
a controller for controlling system state transitions, which can be actuated by the operator;
a control unit operatively connected to both the master device and the slave device, and to said controller for controlling system state transitions, configured to control the robotic system by performing the following actions:
defining a first state of the robotic system, corresponding to a state of teleoperation with fully enslaved following, in which the surgical instrument of the slave device, or at least a control point, belonging to or integral with the surgical instrument of the slave device, is enslaved and follows the master device in each of the degrees of freedom of said plurality of N controllable degrees of freedom;
defining a second state of the robotic system, corresponding to a limited teleoperation state, in which the surgical instrument of the slave device, or at least said control point of the surgical instrument of the slave device, is decoupled from the master device with reference to at least one decoupled degree of freedom, and is enslaved to the master device only in a subset of said plurality of N controllable degrees of freedom which excludes said at least one decoupled degree of freedom;
providing, in said robotic system, a controller for controlling system state transitions;
controlling transitions between said first state of the system and second state of the robotic system, by the operator, by actuating said controller for controlling system state transitions;
wherein the plurality of controllable degrees of freedom comprises degrees of freedom of translation and degrees of freedom of orientation,
and wherein said second limited teleoperation state is a state of repositioning of the master device, wherein said at least one decoupled degree of freedom comprises all the degrees of freedom of translation, and therefore said surgical instrument of the slave device, or said at least one control point of the surgical instrument of the slave device, does not follow the master device in translation.

18. A robotic system according to claim 17, wherein said controller for controlling state transitions comprises a control button, or a control pedal, which are pressable and/or kept pressed and/or released by the operator, and wherein the second limited teleoperation state is activated, during the teleoperation, by keeping said control button pressed, and is deactivated by releasing said control button.

19. A robotic system according to claim 17, wherein the system is a robotic system for teleoperated microsurgery, and wherein said surgical instrument of the slave device is a microsurgical instrument.

20. A robotic system according to claim 17, wherein said subset of controllable degrees of freedom comprises at least two degrees of freedom of orientation, and wherein said surgical instrument of the slave device, or the at least one control point of the surgical instrument of the slave device, follows the master device in said at least two degrees of freedom of orientation.

21-37. (canceled)
